(19) United States
(12) Patent Application Publication
(10) Pub. No.: US 2026/0168901 A1
(43) Pub. Date: Jun. 18, 2026

(54) Artificial Intelligence-Based Rock Fracture Prediction Method for Multi-Physics Monitoring

(71) Applicant: Institute of Geology and Geophysics, Chinese Academy of Sciences, Beijing (CN)

(72) Inventors: Shaojiang WU, Beijing (CN); Yibo WANG, Beijing (CN); Jiawen ZHONG, Beijing (CN)

(73) Assignee: Institute of Geology and Geophysics, Chinese Academy of Sciences, Beijing (CN)

(21) Appl. No.: 19/530,047

(22) Filed: Feb. 4, 2026

(30) Foreign Application Priority Data
Jul. 24, 2025 (CN) 202511023642.8

(51) Int. Cl. G01N 3/10; G01N 3/06; G01N 23/046; G01N 23/083; G01N 33/24

(52) U.S. Cl. CPC G01N 3/10; G01N 3/066; G01N 23/046; G01N 23/083; G01N 33/24; G01N 2203/0064; G01N 2203/0067; G01N 2203/0658

(57) ABSTRACT

This invention provides an artificial intelligence-based multi-physics monitoring method for predicting rock fracture, which adaptively integrates and analyzes multi-physical monitoring results to predict the rock fracture development process. This method will provide real-time guidance information for actual shale gas hydraulic fracturing reservoir modification field operations and provide risk warnings for uncontrollable fractures. The artificial intelligence-based multi-physics monitoring rock fracture prediction method of this invention adaptively integrates and analyzes multi-physical monitoring results to predict the rock fracture development process. These rock fracture development processes and their prediction results provide rock property information under specific pressure conditions, thereby inferring the reservoir mechanical characteristics such as wellhead injection pressure, providing real-time guidance information for applied shale gas hydraulic fracturing reservoir field operations, and providing risk warnings for uncontrollable fractures.

### TECHNICAL FIELD

The present invention relates to “machine learning,” which is “computation based on a specific computational model”; in addition to machine learning, the multiphysics monitoring of the present invention relates to “obtaining images of the interior of an object by emitting ultrasonic waves or acoustic waves through the object”; furthermore, the present invention relates to the field of “geophysics” and also to the field of “extracting oil, gas, water, soluble or fusible substances, or mineral slurry from wells.” Specifically, this invention relates to an artificial intelligence-based multi-physics monitoring method for predicting rock fracture.

### BACKGROUND

Hydraulic fracturing technology involves injecting high-pressure fluid into shale reservoirs to create complex artificial fractures, which can increase reservoir connectivity and improve single-well production. Monitoring and evaluating different stages of hydraulic fracturing reservoir modification is a prerequisite for efficient development and safe production. However, in field monitoring of hydraulic fracturing, the field conditions are complex, the observation system layout is limited, and accurate background information of the actual work area (velocity structure, rock mechanical parameters, etc.) cannot be obtained. At the same time, there are many uncontrollable factors in actual operation, making it difficult to verify the correctness of the fracture inversion results during hydraulic fracturing through field monitoring.

Laboratory rock physics and hydraulic fracturing experiments can provide stable and controllable stress loading and signal measurement conditions. By preparing rock samples from shale gas reservoir outcrops or drill cores, accurate information on rock sample properties under specific pressure conditions can be obtained. This information provides real-time guidance for actual shale gas hydraulic fracturing reservoir stimulation operations and provide risk warnings for uncontrolled fracturing.

Laboratory rock physics hydraulic fracturing experiments can perform multi-physics monitoring (ultrasound, acoustic emission, CT). In laboratory rock physics experiments, multi-physics monitoring such as CT, ultrasound, and acoustic emission can be performed simultaneously. Computed tomography (CT) is an imaging technology that obtains information about the measured object in a non-destructive manner; CT imaging uses an excited X-ray beam to perform tomographic scanning of the rock sample, obtaining information about the medium structure of the rock sample's internal structure. Active source ultrasound uses piezoelectric ceramic sensors (PZT) to excite seismic waves. The seismic waves propagate within the rock sample, and the received seismic waves can be inverted to obtain medium information such as the velocity inside the rock sample. Passive acoustic emission uses piezoelectric ceramic sensors to continuously and passively collect acoustic emission signals generated by rock internal damage. Analysis of these acoustic emission signals provides information about the source parameters related to rock fracturing. In these monitoring systems, piezoelectric ceramic sensors are installed on the rock surface, forming a sensor array in a specific configuration. These PZT sensors utilize a fast automatic switching system to switch between PZT receiving and transmitting functions, allowing them to both passively collect acoustic emission signals and actively generate ultrasonic signals.

Giant earthquakes typically begin with tiny rock fractures at almost a single point, followed by continuous slippage of a complex fault system, extending hundreds of kilometers and radiating enormous energy outwards, causing strong tremors. Hydraulic fracturing uses high-pressure fluid injection to create a network of fractures in the rock, increasing productivity; however, when the fracturing process is improperly designed, the accumulated geostress or energy during hydraulic fracturing may pose a risk of large earthquakes. Therefore, it is necessary to evaluate the hydraulic fracturing process, assess the intrinsic physical relationship between fractures and the fracturing process, and establish methods for fracture prediction to provide a basis for risk control.

In recent years, machine learning methods have developed rapidly and achieved some success in the field of earthquake/fracture prediction. The general approach of machine learning for prediction is as follows: select appropriate seismic observation data, calculate corresponding characteristic indicators as model input variables, and use parameters such as the time, space, and magnitude of the target earthquake as response variables; the model uses the data to establish the relationship between characteristic indicators and seismic events. For seismic observation data and characteristic indicators, data is usually selected from seismological data such as earthquake catalogs and seismic waveform data, and precursory observation data such as surface deformation, electromagnetic fields, ground temperature, gravity, subsurface fluids, and geochemistry. For example, Rouet-Leduc et al. (2018) found a certain correlation pattern between acoustic signals emitted during rock experiment loading and the fault friction coefficient using random forest machine learning. At least in rock experiments, the transient statistical characteristics of seismic signals at any given time can be used to infer the frictional characteristics of the fault zone and the stress state around the fault. Rouet-Leduc et al. (2017) used machine learning to identify hidden signals before rock experiment instability, finding that using only a segment of noisy acoustic signals, they could accurately predict the remaining time until the next frictional instability. Hulbert et al. (2019) used machine learning to predict the time, duration, and magnitude of earthquakes in acoustic emission experiments. The peak slip velocity of slow earthquakes in the laboratory can serve as an important indicator for predicting strong earthquakes.

In general, deep learning has demonstrated advantages such as high performance, scalability, and generalization capabilities in predicting laboratory earthquakes. However, there is no visible research on fracture prediction using multi-physics monitoring data from hydraulic fracturing experiments

### SUMMARY

The objective of the present invention is to at least partially overcome the shortcomings of the prior art and to provide an artificial intelligence-based multi-physics monitoring method for rock fracture prediction.

This method adaptively integrates and analyzes multi-physical monitoring results and predicts the rock fracture development process, providing real-time guidance information for actual gas hydraulic fracturing reservoir stimulation operations and providing risk warnings for uncontrolled fracturing.

Another objective of the present invention is to provide an artificial intelligence-based multi-physics monitoring method for rock fracture prediction that can predict the fracture development process during hydraulic fracturing, thereby predicting the fracture propagation process.

Another objective of the present invention is to provide an artificial intelligence-based multi-physics monitoring method for rock fracture prediction, providing safety warnings for uncontrolled fracturing during actual hydraulic fracturing reservoir stimulation operations.

To achieve the above objectives or one of the above objectives, the technical solution of the present invention is as follows:
An artificial intelligence-based multi-physics monitoring method for rock fracture prediction, the method comprising:
Selecting rock samples from actual shale gas reservoir outcrops or reservoir stimulation drilling cores, conducting laboratory rock physical hydraulic fracturing experiments, and performing multi-physics monitoring;
Constructing a training dataset: conducting the experiments on different rock samples to obtain data, forming a total dataset; the data includes: CT structural images with piezoelectric ceramic sensor metal artifacts removed, rock fusion fracture images, acoustic emission location distribution maps, ultrasonic inversion velocity maps, b-values calculated based on acoustic emission events divided into time segments, axial pressure curves, confining pressure curves, injection pressure curves, injection rate curves, and injection volume curves;
Constructing a fracture prediction module, the fracture prediction module being constructed based on a sequence-to-sequence framework;
Training the detection network using the training dataset;
Preprocessing all actual monitored data using the same steps, inputting the observed data before time t, and obtaining the predicted fracture result at time t+1;

Updating the dataset and the fracture prediction module. According to a preferred embodiment of the present invention, the step of “conducting laboratory rock physics hydraulic fracturing experiments and performing multi-physics monitoring” includes:
Sampling rock samples of different specifications according to the research objectives, covering the rock samples with pre-fabricated rubber sleeves, which have multiple probe holes, with no piezoelectric ceramic sensors attached to the holes;
Collecting CT data of the rock samples without attached piezoelectric ceramic sensors using an laboratory hydraulic fracturing experimental device. This CT data does not contain metal artifacts from the piezoelectric ceramic sensors and is used as the first stage CT data;
Placing the piezoelectric ceramic sensors into the probe holes and bonding the piezoelectric ceramic sensors to the surface of the rock samples;

Collecting CT data of the rock samples with attached piezoelectric ceramic sensors using the laboratory hydraulic fracturing experimental device, and using the CT data to calibrate the spatial position of the piezoelectric ceramic sensors. This CT data contains metal artifacts from the piezoelectric ceramic sensors and is used as the second stage CT data;
Applying different loading strategies to the rock samples. The loading stages include an isotropic loading stage, an axial pressure increase stage, a water injection and pore pressure increase stage, and a pressure unloading stage; during the water injection and pore pressure increase stage, collecting several sets of CT data at set time intervals. This CT data contains metal artifacts from the piezoelectric ceramic sensors and is used as the third stage CT data; during the pressure unloading stage, collecting one set of CT data. This CT data contains metal artifacts from the piezoelectric ceramic sensors and is used as the fourth stage CT data; then, removing the piezoelectric ceramic sensors from the rock samples and collecting another set of CT data. This CT data does not contain metal artifacts from the piezoelectric ceramic sensors and is used as the fifth stage CT data;
Processing all the collected CT data. According to a preferred embodiment of the present invention, the rock samples have the following specifications: cylindrical shape with a diameter of 50 mm and a length of 125 mm;
The rock samples include two types: ordinary rock samples and interfering rock samples;
The ordinary rock samples include two types of rocks: sandstone and shale; they are divided into 5 categories based on the presence and direction of bedding, namely bedding directions of 0±20°, 45±20°, 90±20°, 135±20°, and homogeneous medium without bedding; they are also divided into 2 categories based on axial compressive strength, namely 50±10 MPa and 90±10 MPa; 5 rock samples are selected for each category, totaling 100 rock samples;

The interfering rock samples include two types of rocks: sandstone and shale, which are homogeneous media without bedding, and the rock samples contain sediments; 5 rock samples are selected for each type, totaling 10 rock samples.

According to a preferred embodiment of the present invention, the dataset is generated from real rock sample experimental data; the data is augmented to improve the generality of the dataset; all data undergoes the same preprocessing steps.

According to a preferred embodiment of the present invention, the generation of real rock sample experimental data includes:
Rock samples were prepared from actual shale gas reservoir outcrops or core samples from reservoir stimulation wells. Standard rock samples were then used for laboratory rock physics and hydraulic fracturing experiments, and multiple experiments were conducted to obtain multiple sets of experimental data;
For any k-th experiment, the start time of the water injection and pore pressure increase stage is set as the initial value TO, the total duration is Tn, and data is collected every dt seconds, resulting in Nr data points; in all the multiple sets of experimental data, different rock types lead to differences in the experimental process, thus Tn and Nr will vary.

According to a preferred embodiment of the present invention, the step of “augmenting the data” includes:
In the acoustic emission location distribution map at any time t, adding random interference to the calculated source location information of the acoustic emission source, and regenerating the acoustic emission location distribution map;
In the b-value at any time t, adding random interference to the excitation time of the calculated acoustic emission source information, and regenerating the b-value. According to a preferred embodiment of the present invention, in the “training the detection network using the training dataset” step, the dataset is divided into a training set and a test set in a ratio of 8:2; the fracture prediction module uses a stochastic gradient descent optimization method, with a dynamic learning rate, an initial value of 0.0001, decreasing by half every 50 iterations, a batch size of 40, and 200 iterations; the training of the fracture prediction module is performed on a GPU image processing unit.

According to a preferred embodiment of the present invention, the training dataset is updated when any of the following conditions are triggered:
When the time difference between the first appearance of a crack in the actual results and the predicted results during the actual monitoring process is greater than 10 seconds; or
When the similarity of cracks in adjacent 5-second intervals in the actual results and predicted results during the actual monitoring process is less than 50%.

According to a preferred embodiment of the present invention, the update of the fracture prediction module is triggered when the following conditions are met:
When the number of newly added experiments in the dataset is greater than 5; or
When the proportion of newly added experiments in the dataset is greater than 20%.

According to a preferred embodiment of the present invention, the laboratory hydraulic fracturing experimental device includes a pressure vessel, a loading system, an acoustic emission counting and waveform acquisition system, and a CT monitoring system.

According to a preferred embodiment of the present invention, during the pressurization of rock samples using different loading strategies, active source ultrasonic data is collected at set times. During active source ultrasonic monitoring, some piezoelectric ceramic sensors are used as transmitting probes to excite ultrasonic signals, and the remaining piezoelectric ceramic sensors are used as receiving probes to receive ultrasonic signals;

During the pressurization of rock samples using different loading strategies, the piezoelectric ceramic sensors, in addition to being used as transmitting and receiving probes during active source ultrasonic acquisition, are used as receiving probes to receive acoustic emission signals generated by changes in the rock sample at other times.

The artificial intelligence-based multi-physics monitoring and rock fracture prediction method of this invention adaptively integrates and analyzes multi-physical monitoring results to predict the rock fracture development process. These rock fracture development processes and their prediction results at different times provide information about rock properties under specific pressure conditions, thereby inferring the reservoir mechanical characteristics under specific construction conditions such as wellhead injection pressure. This method will provide real-time guidance for actual shale gas hydraulic fracturing reservoir stimulation field operations and provide risk warnings for uncontrolled fracturing.

### BRIEF DESCRIPTION OF DRAWINGS

FIG. 1 shows the arrangement of the rock sample and piezoelectric ceramic sensors according to an embodiment of the present invention, with the rock sample on the left and the arrangement of PZT on the surface of the rock sample on the right;

FIG. 2 shows the modular architecture of the artificial intelligence-based multi-physics monitoring and rock fracture prediction method according to an embodiment of the present invention;

FIG. 3 is a schematic diagram of the structure of the fracture prediction module according to an embodiment of the present invention; and

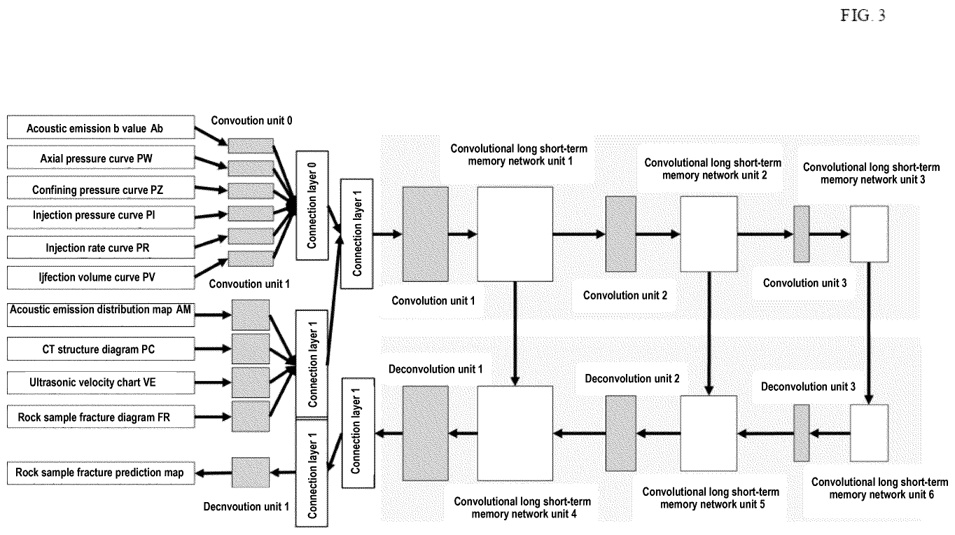

FIG. 4 is a schematic diagram of the structure of the ConvLSTM unit and Conv/DeConv unit in the fracture prediction module according to an embodiment of the present invention.

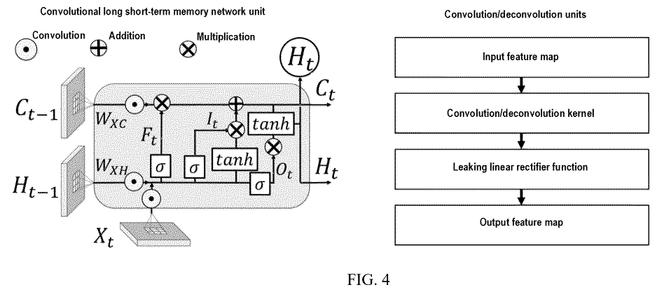

### DETAILED DESCRIPTION OF THE EMBODIMENTS

The exemplary embodiments of the present invention are described in detail below with reference to the accompanying drawings, where the same or similar reference numerals represent the same or similar elements. Furthermore, in the following detailed description, for ease of explanation, numerous specific details are set forth to provide a thorough understanding of the disclosed embodiments. However, it will be apparent that one or more embodiments may be practiced without these specific details. In other instances, well-known structures and devices are shown in diagrammatic form to simplify the drawings.

This application proposes an artificial intelligence-based method for predicting rock fracture in laboratory hydraulic fracturing physical experiments using multi-physics monitoring data. This method is based on a multi-input multi-layer ConvLSTM (Convolutional Long Short-Term Memory) network, whose spatio-temporal coupling structure can extract the temporal and spatial correlation features within the multi-physical data. First, the multi-physics data (loading parameters, acoustic emission information, ultrasonic information, and CT images) are set to the same time axis intervals; then, the discrete information within each interval is integrated and transformed into spatial information at that moment (for example, discrete acoustic emission information is converted into an acoustic emission distribution map); by inputting the multi-physics data from multiple consecutive time points into the prediction network, the fracture development process during hydraulic fracturing is obtained, and rock fracture can be predicted.

This method can adaptively extract the intrinsic temporal and spatial correlations of multi-physical monitoring data, predict the fracture development process during hydraulic fracturing, and thus predict the fracture process, providing important theoretical reference information for unconventional shale gas development.

Artificial Intelligence (AI) is the theory, methods, techniques, and application systems that use digital computers or machines controlled by digital computers to simulate, extend, and expand human intelligence, perceive the environment, acquire knowledge, and use knowledge to obtain optimal results. Machine Learning (ML) is a multidisciplinary field involving probability theory, statistics, approximation theory, convex analysis, algorithm complexity theory, and many other disciplines. It specifically studies how computers simulate or implement human learning behavior to acquire new knowledge or skills and reorganize existing knowledge structures to continuously improve their performance. Machine learning is the core of artificial intelligence and the fundamental way to make computers intelligent; its applications are widespread in various fields of artificial intelligence. Machine learning and deep learning usually include techniques such as artificial neural networks, belief networks, reinforcement learning, transfer learning, inductive learning, and supervised learning. The following describes the specific process of the artificial intelligence-based multi-physics monitoring and rock fracture prediction method according to an embodiment of the present invention.

### 1. Basic Information of Rock Physical Experiments Involved in This Invention

### 1.1 Experimental Sample Preparation:

Rock samples are typically obtained from outcrops of actual shale gas reservoirs or from core samples of reservoir stimulation wells. The dimensions can vary depending on the specific research objectives; conventional laboratory hydraulic fracturing experiments use cylindrical samples with the following specifications: 50 mm in diameter and 125 mm in length.

Two types of rock samples are set: ordinary rock samples and interfering rock samples:

Ordinary rock sample types include: rock type (2 types, such as sandstone and shale), with different bedding directions (bedding directions are approximately 0±20°, 45±20°, 90±20°, 135±20°, and homogeneous media without obvious bedding, 5 types in total), and different axial compressive strengths (50±10 MPa, 90±10 MPa, 2 types in total); 5 rock samples are selected for each type, totaling 100 rock samples.

Interfering rock sample types include: rock type (2 types, such as sandstone and shale), homogeneous media without obvious bedding, and the rock samples contain obvious sediments, such as heavy mineral impurities and clastic particles. These sediments are internal characteristics of the rock sample and will be visible in CT images regardless of whether PZT probes are attached to the surface, increasing the universality of the rock sample. 5 rock samples are selected for each type, totaling 10 rock samples.

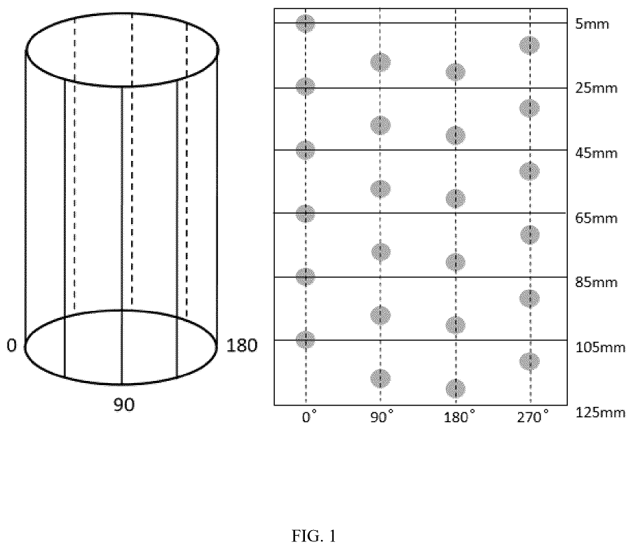

A pre-fabricated rubber sleeve is used to cover the rock sample. The rubber sleeve has 24 PZT probe holes (not limited to 24, this number depends on the design of the rubber sleeve). The PZT probes are placed in the holes, and the bottom of the PZT probes are glued to the surface of the rock sample. The positions of the PZT probe holes on the rubber sleeve are shown in FIG. 1, arranged in four columns along 0°, 90°, 180°, and 270° of the rock sample. The height of the probes in each column is different, ensuring that the probes in the four columns do not appear at the same height simultaneously. Since PZT is made of metal, it will produce serious metal artifacts on the rock sample surface, affecting the CT imaging quality of the rock sample; this design will reduce the metal influence caused by multiple PZT probes at any given height (as shown in FIG. 1).

### 1.2 Experimental System Configuration:

The experimental system is an laboratory hydraulic fracturing experimental device, mainly consisting of a pressure vessel, a loading system, and an acoustic emission counting and waveform acquisition system.

(1) The pressure vessel is a high-pressure resistant metal container, in which the prepared rock sample is placed.

(2) The loading system generally uses a triaxial compression stress loading system. This system can provide axial pressure, injection pressure, and confining pressure to the inside of the pressure vessel, used to simulate the in-situ formation conditions of the rock sample.

(3) The acoustic emission counting and waveform acquisition system consists of PZT sensors, preamplifiers, and high-speed acquisition equipment.

(4) The CT monitoring system consists of a CT scanner, meeting a field of view greater than 125 mm, with a scanning mode of planar scanning, typically a 256-slice CT scanner.

### 1.3 Experimental Procedure:

The experimental system can adopt different loading strategies according to the actual research objectives. The experimental procedure in this application is as follows:

(1) Place the rock sample in a rubber sleeve and load it into the pressure vessel; place it on the CT scanner and acquire CT data once. The CT data acquired in this process does not include interference from PZT metal artifacts.

(2) Remove the rock sample from the container, and attach PZT sensors (or PZT probes) to the PZT sensor holes in the rubber sleeve outside the rock sample.

(3) Place the rock sample with attached PZT sensors (or PZT probes) into the pressure vessel; place it on the CT scanner and acquire CT data once. The CT data acquired in this process includes interference from PZT metal artifacts. Calibrate the spatial position Sta (x,y,z) of all sensors (because the PZT sensor hole diameter is larger than the sensor diameter, there will be spatial deviations when installing the PZT sensors in each experiment, so the position needs to be accurately extracted from the CT image after installing the PZT sensors).

(4) Isotropic loading stage (confining pressure).

(5) Increasing axial pressure stage (axial pressure).

(6) Water injection to increase pore pressure stage (injection pressure), until the rock fracture stage and unloading stage. During this process, the CT scanner is turned on, and CT data is acquired according to the set time. The CT data acquired in this process includes interference from PZT metal artifacts.

(7) Pressure unloading stage (injection pressure, confining pressure, and axial pressure are unloaded to 0): CT data is acquired once. The CT data acquired during this process includes interference from PZT metal artifacts (after the rock sample fractures, it contains internal cracks). The rock sample is removed from the container, and the PZT probes attached to the PZT probe holes in the rubber sleeve outside the rock sample are removed. The rock sample is then placed back into the pressure vessel and placed on the CT scanner to acquire CT data. The CT data acquired during this process does not include interference from PZT metal artifacts.

During the above processes (4)-(6), active source ultrasonic data is acquired according to the set time. During active source ultrasonic monitoring, some PZT probes act as transmitting probes (excitation probes) to excite ultrasonic signals, while the remaining probes act as receiving probes to receive ultrasonic signals.

During the above processes (4)-(6), due to the application of external force, acoustic emission events will occur. Except when acting as transmitting probes during active source ultrasonic acquisition, the PZT probes act as receiving probes for the rest of the time, receiving acoustic emission signals generated by changes in the rock sample.

### 1.4, Main Data Analysis Content:

The CT data processing flow includes: data acquisition, CT imaging, PZT metal artifact suppression, and joint analysis with acoustic emission results.

The acoustic emission data processing flow includes: effective event picking (picking data segments with clear phases), first arrival picking (extracting the first arrival time of the waveform in the effective event data), source location (obtaining the source location of the event based on the picked first arrival time and the spatial position of the probes), source mechanism analysis (inverting the source mechanism of the event based on the waveform of the event to obtain the fracture characteristics of the event), magnitude calculation (inverting the magnitude of the event based on the waveform of the event), stress field analysis (based on the waveform of the event or the source mechanism of the event), etc.

The ultrasonic data processing flow includes: ultrasonic event identification (identifying the signals received by other channels based on the ultrasonic excitation time), ultrasonic first arrival picking (obtaining the first arrival time of the received signal), and velocity inversion analysis (inverting to obtain the velocity model of the rock sample based on the first arrival time, waveform, and other information of the signal).

### 2. Constructing the Training Dataset:

### 2.1 Dataset Overview.

Using the ordinary rock samples prepared in 1.1, and completing the experimental process described in 1.3, a dataset for a single experiment can be obtained; by repeating this experiment Nₖ times using different ordinary rock samples from 1.1, a total dataset is formed.

The k-th experiment includes the following data:

(1) CT structural image DCTt(N₁,N₂,N₃,Nₜ) with PZT metal artifacts removed, a four-dimensional dataset including three spatial dimensions N₁,N₂,N₃, and one time dimension Nₜ.

(2) Rock fracture fusion image FRt(N₁,N₂,N₃,Nₜ), a four-dimensional dataset including three spatial dimensions N₁,N₂,N₃₃, and one time dimension Nₜ.

(3) Acoustic emission location distribution map DAEt(N₁,N₂,N₃,Nₜ), a four-dimensional dataset including three spatial dimensions N₁,N₂,N₃, and one time dimension Nₜ.

(4) Ultrasonic inversion velocity map DVEt(N₁,N₂,N₃,Nₜ), a four-dimensional dataset including three spatial dimensions N₁,N₂,N₃, and one time dimension Nₜ.

(5) b-value Ab(Nₜ) calculated based on acoustic emission events divided into time intervals, a one-dimensional time series data.

(6) Axial pressure curve PW(Nₜ), a one-dimensional time series data.

(7) Confining pressure curve PW(Nₜ), a one-dimensional time series data.

(8) Injection pressure curve PI(Nₜ), a one-dimensional time series data.

(9) Injection rate curve PR(Nₜ), a one-dimensional time series data.

(10) Injection volume curve PV(Nₜ), a one-dimensional time series data.

2.2 Dataset Generation.

The dataset is generated from real rock sample experimental data.

Using the ordinary rock samples prepared in 1.1, and employing the experimental process described in 1.3,Nₖ experiments are completed to obtain Nₖ sets of experimental data. For each k-th experiment, process 1.3 (6) starts at initial time T0, with a total duration of Tn. Ultrasound data is collected every dt seconds, resulting in Nr data points. CT data is also collected every dt seconds, resulting in Nₜ data points. Across all Nₖ experiments, differences in rock types lead to variations in the experimental process, and consequently, variations in Tn and Nₜ.

(1) CT Structural Image with PZT Metal Artifact Removal (DCTt)

At time T0+dt*t, the original CT image containing PZT metal artifacts is acquired. A metal artifact suppression method (e.g., matched subtraction method) is used to remove the metal artifacts from the CT image. This process is repeated for Nₜ time points to obtain the data for all time points, forming DCTt(N₁,N₂,N₃,Nₜ).

### (2) Rock Fracture Fusion Image (FRt)

At time T0+dt*t, the current CT structural image, acoustic emission distribution map, and other physical field information are integrated and interpreted to obtain the fracture image. This process is repeated for Nr time points to obtain the data for all time points, forming FRt(N₁,N₂,N₃,Nₜ).

### (3) Acoustic Emission Location Distribution Map (DAEt)

Acoustic emission events are monitored during the time interval from T0 to T0+dt*t. The source parameters of all acoustic emission signals are calculated. Selecting a magnitude range from Mg1 to Mg2, the source parameters of any acoustic emission event are (x,y,z,it,Mg), where XYZ represents the location, it is the excitation time, and Mg is the magnitude.

The magnitude is converted from the exponential domain to the decimal domain using the formula: EMg=log Mg−(Mg1). The magnitude in the decimal domain is then normalized using the formula:

$$EVMg = \frac{EMg - Mg 1}{Mg 2 - Mg 1} .$$

Therefore, the information for any acoustic emission event is (x,y,z,it,EVMg). The acoustic emission event information is transformed into the data space (x,y,z)=EVMg, where within (N₁,N₂,N₃), each acoustic emission event is represented by its source location (x,y,z) as coordinates and its magnitude EVMg as the coordinate amplitude. Each acoustic emission event in the data space is then transformed into a probability distribution map. For any event in the data space (N₁,N₂,N₃), Gaussian smoothing is performed with a standard deviation of 1 and a window of 5×5×5 to obtain the Gaussian distribution map of that event in the data space.

Using the initial value TO as a reference, the Gaussian distribution maps of all acoustic emission events in the time period from T0 to T0+dt*t are added together to obtain the acoustic emission distribution map at time T0+dt*t. This process is repeated for Nₜ time points to obtain the data DAEt(N₁,N₂,N₃,Nₜ) for all time points.

(4) Time-Varying Velocity Map of Ultrasound (Velocity Map from Ultrasonic Inversion) DVEt

Using the initial value TO as a reference, all ultrasonic data collected in the time period from T0+dt*(t−1) to T0+dt*t are used with a velocity inversion method (e.g., linear inversion) to obtain the velocity map of the rock sample data space at time T0+dt*t. This process is repeated for Nₜ time points to obtain the data DVEt(N₁,N₂,N₃,Nₜ) for all time points.

(5) b-Value Ab Calculated Based on Acoustic Emission Events Divided into Time Segments

Using the initial value TO as a reference, the method in 2.3 (1) is used to obtain the b-value Ab (t) based on the magnitude Mg for the data in the time period from T0+dt*(t−1) to T0+dt*t. This process is repeated for all time points to obtain Ab(Nₜ).

### (6) Axial Pressure Curve PW

Using the initial value TO as a reference, select the continuously monitored axial pressure curve and remove outliers; set the axial pressure range from 0 to 100 MPa, normalize the actual axial pressure curve, and use the value at time T0+dt*t as PW (t), iterating through time to obtain PW(Nₜ).

### (7) Confining Pressure Curve PZ

Using the initial value TO as a reference, select the continuously monitored confining pressure curve and remove outliers; set the confining pressure range from 0 to 50 MPa, normalize the actual confining pressure curve, and use the value at time T0+dt*t as PZ(t), iterating through time to obtain PZ(Nr).

### (8) Injection Pressure Curve PI

Using the initial value TO as a reference, select the continuously monitored injection pressure curve and remove outliers; set the injection pressure range from 0 to 50 MPa, normalize the actual injection pressure curve, and use the value at time T0+dt*t as PI(t), iterating through time to obtain PI(Nₜ).

### (9) Injection Rate Curve PR

Using the initial value TO as a reference, select the continuously monitored injection rate curve and remove outliers; set the injection rate range from 0 to 5 ml/min, normalize the actual injection rate curve, and use the value at time T0+dt*t as PR(t), iterating through time to obtain PR(Nₜ).

### (10) Injection Volume Curve PV

Using the initial value TO as a reference, select the continuously monitored injection volume curve and remove outliers; set the injection volume range from 0 to 1000 ML, normalize the actual injection volume curve, and use the value at time T0+dt*t as PV(t), iterating through time to obtain PV(Nₜ).

### 2.3, Calculation Methods for Relevant Data

(1) b-Value Calculation Method

The b-value reflects the relationship between magnitude and frequency. A smaller b-value indicates a higher frequency of larger magnitude earthquakes. It is generally believed that the b-value is related to the uniformity of the medium and the stress level. The empirical relationship between magnitude and frequency should satisfy the following exponential distribution:

$$ln N = a - bM$$

Using the maximum likelihood estimation method, the formula for calculating the value of bis:

$${b}_{i} = \frac{n ln e}{{\sum }_{j = 0}^{n - 1} ( {M}_{i - j} - {M}_{st} )}$$

Where Mᵢ₋ⱼ is the magnitude of the j-th earthquake, and Mₛₜ is the lower limit magnitude.

### 2.4. Data Augmentation.

(1) In DAEt(t) at any time t in 2.2(3), random noise is added to the calculated source location (x, y, z) of the acoustic emission source information, and the relevant data DAEt(Nₜ) is regenerated.

(2) In Ab(t) at any time t in 2.2(5), random noise is added to the excitation time (it) of the calculated acoustic emission source information, and the relevant data Ab(Nₜ) is regenerated.

### 3. Construction of the Fracture Prediction Method:

### 3.1. Module Architecture.

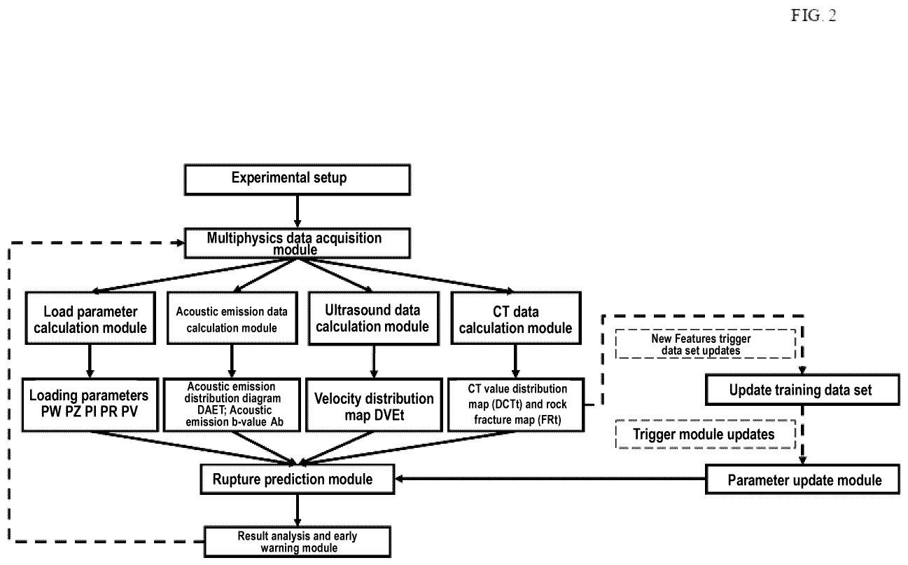

The fracture prediction method is shown in FIG. 2, and includes the experimental setup, multi-physics data acquisition module, data processing module, fracture prediction module, results analysis and early warning module, and parameter update module.

The experimental setup and multi-physics data acquisition module mainly include the experimental part in 1.3, corresponding to that section.

The data processing module includes the calculation module for multi-physics data, including the calculation of loading parameters, acoustic emission data, CT data, and ultrasonic data, mainly including the methods involved in dataset generation in 2.2.

The fracture prediction module contains the main structure of the fracture prediction method, mainly including section 3.2.

The results analysis and early warning module mainly includes the application part of the fracture prediction method, mainly including section 3.3.

### 3.2. Fracture Prediction Module

The fracture prediction module is constructed based on a sequence-to-sequence (Seq-to-Seq) framework.

The input part consists of data from time 1 to Nₜ, with two types of data sizes. FR, PC, AM, and VE are four-dimensional data (N₁,N₂,N₃,Nₜ); a convolutional layer Conv1 unit is used for preliminary feature extraction; a connection layer Dense0 is used to connect these four extracted features. PW, PZ, PI, PR, and PV are one-dimensional data (Nₜ), and a convolutional layer Conv0 unit is used for preliminary feature extraction; a connection layer Dense1 is used to connect these six extracted features. The above data is further connected using a Dense1 layer to form input feature data in the (N₁,N₂,N₃,Nₜ) format.

In the output section, the output is a rupture prediction map for time Nₜ+1. The actual observed rupture map and the predicted rupture map at time Nₜ+1 are used to calculate the neural network error using the mean squared error (MSE) as the loss function.

In the encoder section, the convolutional layer Conv further extracts features from these input feature data. The feature maps then enter the ConvLSTM unit to extract the temporal and spatial correlations of the feature maps. The ConvLSTM unit is divided into two data streams: the rightward output serves as the input for subsequent convolutional units, and the downward output serves as the output state for the corresponding input of the next ConvLSTM unit. The convolutional layer Conv units and ConvLSTM units are alternately repeated 3 times, from Conv1 to Conv3 and ConvLSTM1 to ConvLSTM3, gradually extracting and encoding information from the seismogram at different scales. This encoding process obtains the spatio-temporal correlation features in the seismic distribution map.

In the decoder section, the features obtained from the encoder are input into the ConvLSTM unit for decoding. The ConvLSTM unit ensures that the decoding process can simultaneously focus on the current state of the data and consider previous states. Then the data enters the deconvolutional DeConv unit to increase the image size, maintaining the same image format at the same scale in both the encoding and decoding parts. Similarly, the deconvolutional layer DeConv units and ConvLSTM units are alternately repeated 3 times, from DeConv1 to DeConv3 and DeConvLSTM1 to ConvLSTM3.

In the above encoding and decoding process, the ConvLSTM operation does not change the image size, while the Conv and DeConv unit operations change the image size. The Conv unit includes a convolutional layer, activation function, etc.

The DeConv unit includes a deconvolutional layer, activation function, etc.

The calculation formulas involved in the ConvLSTM unit (this calculation part is a general module):

$$Input gate : {i}_{t} = σ ( {W}_{xi} ⊙ {X}_{t} + {W}_{hi} ⊙ {H}_{t - 1} + {b}_{i} )$$

$$Forget gate : {i}_{t} = σ ( {W}_{xi} ⊙ {X}_{t} + {W}_{hi} ⊙ {H}_{t - 1} + {b}_{i} )$$

$$Candidate state : {C}_{t} = tanh ( {W}_{xc} ⊙ {X}_{t} + {W}_{hc} ⊙ {H}_{t - 1} + {b}_{c} )$$

$$Cell state : {C}_{t} = {f}_{t} ⊗ {C}_{t - 1} + {i}_{t} ⊗ {\tilde{C}}_{t}$$

$$Output gate : {o}_{t} = σ ( {W}_{xo} ⊙ {X}_{t} + {W}_{ho} ⊙ {H}_{t - 1} + {b}_{o} )$$

$$Hidden state : {H}_{t} = {o}_{t} ⊗ tanh ( {C}_{t} )$$

where └ denotes convolution operation, ⊗ is element-wise multiplication, σ is the Sigmoid activation function, tanh is the hyperbolic tangent activation function, W_{xf},Wₓᵢ,W_{xC},Wₓₒ,W_{hf},Wₕᵢ,W_{hC},Wₕₒ are weight matrices, and b_{f},bᵢ,b_{C},bₒ are bias vectors. All intermediate states Cₜ, Hₜ maintain a 3D structure to achieve continuous modeling of spatiotemporal evolution.

During training, the inputs on the left side are: CT images without PZT artifact interference DCTt(N₁,N₂,N₃,t−n:t); acoustic emission location distribution map DAEt(N₁,N₂,N₃,t−n:t); ultrasonic inversion velocity map DVEt(N₁,N₂,N₃,t−n:t); b-value of acoustic emission events

Ab(t−n:t); axial pressure curve PW(t−n:t); confining pressure curve PZ(t−n:t); injection pressure curve PI(t−n:t); injection rate curve PR(t−n:t); injection volume curve PV(t−n:t); and rock fracture map FRt(N₁,N₂,N₃,t−n:t). The output on the right side is the rock fracture map FRt(N₁,N₂,N₃, t+1) at the next time step. The fracture prediction module uses a Mean Squared Error (MSE) loss function to calculate the residual between the predicted and observed values.

### 3.3, Results Analysis and Early Warning Module

At time step Nr, the fracture prediction module outputs the predicted fracture value. Based on the predicted fracture value, it provides decision-making information to the multi-physics data acquisition module, including experimental operations such as stopping the experiment and adjusting parameters.

### 4. Training the Detection Network Using the Training Dataset

4.1, The dataset is divided into a training set and a test set, with a ratio of 8:2.
4.2, This fracture prediction module uses the stochastic gradient descent optimization method; a dynamic learning rate is set, with an initial value of 0.0001, decreasing by half every 50 iterations. The batch size is set to 40; the number of iterations is 200.
4.3, The fracture prediction module training is performed on a GPU image processing unit.

### 5. Actual Monitoring Data Analysis and Processing:

### 5.1, Actual Data Preprocessing:

Rock samples were prepared from actual shale gas reservoir outcrops or drill cores. All real-time monitoring data underwent the same preprocessing steps. Real-time data for FRt, DCTt, DAEt, and DVEt; PW, PZ, PI, PR, and PV were obtained.

### 5.2, Actual Data Analysis.

Input the observed data before the current time t to obtain the predicted fracture result FRt at time t+1.

When any of the following conditions are met, the training dataset is updated, i.e., the data from this experiment is added to the dataset according to the data generation method in 2.2.

(1) When, during the actual monitoring process in 5, the time difference between the first appearance of a crack in the actual results and the predicted results is greater than 10 seconds;

(2) When, during the actual monitoring process in 5, the similarity of cracks in adjacent 5-second intervals between the actual results and the predicted results is less than 50%;

### 6. Parameter Update Module and Parameter Update

6.1, Module Update Mechanism. The module triggers an update when the following conditions are met:
(1) When the number of new experiments added to the dataset in 5.2 is greater than 5;
(2) When the proportion of new experiments added to the dataset in 5.2 is greater than 20%;
6.2, Parameter Update Method.

The entire fracture prediction module is trained using the updated dataset.
6.3. Updating the fracture prediction module parameters.

The artificial intelligence-based multi-physics monitoring method for rock fracture prediction of the present invention adaptively integrates and analyzes multi-physical monitoring results to predict the rock fracture development process, predicting fractures and their fracture process. Furthermore, the artificial intelligence-based multi-physics monitoring method for rock fracture prediction of the present invention constructs a multi-physical field-based rock fracture prediction module, which will provide early warning for uncontrollable fractures and provide a reference for safe development in actual hydraulic fracturing.

The artificial intelligence-based multi-physics monitoring and rock fracture prediction method of this invention adaptively integrates and analyzes multi-physical monitoring results to predict the rock fracture development process. These rock fracture development processes and their prediction results at different times provide information about rock properties under specific pressure conditions, thereby inferring the reservoir mechanical characteristics under specific construction conditions, such as wellhead injection pressure. This method will provide real-time guidance for actual shale gas hydraulic fracturing reservoir stimulation field operations and provide risk warnings for uncontrolled fracturing.

Although embodiments of the present invention have been shown and described, it will be understood by those skilled in the art that changes may be made to these embodiments without departing from the principles and spirit of the invention. The scope of the invention is defined by the appended claims and their equivalents.

1. An artificial intelligence-based multi-physics monitoring method for rock fracture prediction, comprising conducting laboratory rock physical hydraulic fracturing experiments and performing multi-physics monitoring as follows:
constructing a training dataset, by performing the aforementioned experiments on different rock samples to obtain data, forming a total dataset, wherein the data includes: CT structural images with piezoelectric ceramic sensor metal artifacts removed, rock fusion fracture images, acoustic emission location distribution maps, ultrasonic inversion velocity maps, b-values calculated based on acoustic emission events divided into time segments, axial pressure curves, confining pressure curves, injection pressure curves, injection rate curves, and injection volume curves;
constructing a fracture prediction module, which is built based on a multi-layer ConvLSTM network;
training the detection network using the training dataset;
preprocessing all actually monitored data using the same steps, inputting observation data before time t, and obtaining the predicted fracture result at time t+1;
updating the dataset and the fracture prediction module.

2. The artificial intelligence-based multi-physics monitoring method for rock fracture prediction according to claim 1, characterized in that the step of “conducting laboratory rock physical hydraulic fracturing experiments and performing multi-physics monitoring” includes:
sampling rock samples of different specifications according to the research objectives, covering the rock samples with pre-fabricated rubber sleeves having multiple probe holes, wherein the holes are configured to be fitted with piezoelectric ceramic sensors;
collecting CT data of the rock samples without piezoelectric ceramic sensors using an laboratory hydraulic fracturing experimental device, wherein the CT data does not contain metal artifacts from the piezoelectric ceramic sensors and is used as the first stage CT data;
placing the piezoelectric ceramic sensors into the probe holes and bonding the piezoelectric ceramic sensors to the surface of the rock samples;
collecting CT data of the rock samples with the piezoelectric ceramic sensors attached using the laboratory hydraulic fracturing experimental device, and using the CT data to calibrate the spatial position of the piezoelectric ceramic sensors, wherein the CT data contains metal artifacts from the piezoelectric ceramic sensors and is used as the second stage CT data;
applying different loading strategies to the rock samples, wherein loading stages include an isotropic loading stage, an increasing axial pressure stage, a water injection and pore pressure increase stage, and a pressure unloading stage; wherein, during the water injection and pore pressure increase stage, several sets of CT data are collected at set time intervals, wherein this CT data contains metal artifacts from the piezoelectric ceramic sensors and is used as the third stage CT data; during the pressure unloading stage, collecting one set of CT data, wherein this CT data contains metal artifacts from the piezoelectric ceramic sensors and is used as the fourth stage CT data; and, removing the piezoelectric ceramic sensors from the rock samples and collecting another set of CT data, wherein this CT data does not contain metal artifacts from the piezoelectric ceramic sensors and is used as the fifth stage CT data;
processing all the collected CT data.

3. The artificial intelligence-based multi-physics monitoring method for rock fracture prediction according to claim 2, characterized by:
the rock samples have the following specifications: diameter of 50 mm, length of 125 mm, cylindrical shape;
the rock samples include two types: ordinary rock samples and interfering rock samples;
wherein the ordinary rock samples include two types of rocks: sandstone and shale; they are divided into 5 categories based on the presence and direction of bedding, namely bedding directions of 0±20°, 45±20°, 90±20°, 135±20°, and homogeneous medium without bedding; they are further divided into 2 categories based on axial compressive strength, namely 50±10 MPa and 90±10 MPa; and five rock samples are selected from each category, totaling 100 rock samples;
wherein the interfering rock samples include two types of rocks: sandstone and shale, which are homogeneous media without bedding, and the rock samples contain sediments; and five rock samples are selected from each category, totaling 10 rock samples.

4. The artificial intelligence-based multi-physics monitoring method for rock fracture prediction according to claim 3, characterized in that:
the dataset is generated from real rock sample experimental data; data augmentation is performed to improve the generality of the dataset; and all data are preprocessed using the same steps.

5. The artificial intelligence-based multi-physics monitoring method for rock fracture prediction according to claim 4, characterized in that the generation of real rock sample experimental data includes:
rock samples were prepared from actual shale gas reservoir outcrops or core samples from reservoir stimulation wells, and then used for laboratory rock physics and hydraulic fracturing experiments, and multiple experiments were conducted to obtain multiple sets of experimental data;
wherein, for any k-th experiment, the start time of the water injection and pore pressure increase stage is set as the initial value TO, the total duration is Tn, and data is collected every dt seconds, resulting in Nₜ data points; and in all the multiple experimental data, different rock types lead to differences in the experimental process, and thus Tn and Nₜ will vary.

6. The artificial intelligence-based multi-physics monitoring method for rock fracture prediction according to claim 5, wherein the step of “data augmentation” includes:
adding random noise to the source location of the calculated acoustic emission source information in the acoustic emission location distribution map at any time t, and regenerating the acoustic emission location distribution map;
adding random noise to the excitation time of the calculated acoustic emission source information in the b-value at any time t, and regenerating the b-value.

7. The artificial intelligence-based multi-physics monitoring method for rock fracture prediction according to claim 1, wherein:
in the “training the detection network using the training dataset” step, the dataset is divided into a training set and a test set in a ratio of 8:2; the fracture prediction module uses a stochastic gradient descent optimization method, with a dynamic learning rate, an initial value of 0.0001, reduced by half every 50 iterations, a batch size of 40, and 200 iterations; the training of the fracture prediction module is performed on a GPU image processing unit.

8. The artificial intelligence-based multi-physics monitoring method for rock fracture prediction according to claim 1, wherein the training dataset is updated when any of the following conditions are triggered:
when the time difference between the first appearance of a crack in the actual results and the predicted results during actual monitoring is greater than 10 seconds; or
when the similarity of cracks in adjacent 5 seconds between the actual results and the predicted results during actual monitoring is less than 50%.

9. The artificial intelligence-based multi-physics monitoring method for rock fracture prediction according to claim 8, wherein the update of the fracture prediction module is triggered when the following conditions are met:
when the number of newly added experiments in the dataset is greater than 5; or
when the proportion of newly added experiments in the dataset is greater than 20%.

10. The artificial intelligence-based multi-physics monitoring method for rock fracture prediction according to claim 2, wherein:
the laboratory hydraulic fracturing experimental device includes a pressure vessel, a loading system, an acoustic emission counting and waveform acquisition system, and a CT monitoring system.
